(19) United States
(12) Patent Application Publication
(10) Pub. No.: US 2003/0192549 A1
(43) Pub. Date: Oct. 16, 2003

(54) Device for suppressing snoring

(76) Inventor: Georges Boussignac, Antony (FR)

Correspondence Address: STEVENS DAVIS MILLER & MOSHER, LLP, 1615 L STREET, NW, SUITE 850, WASHINGTON, DC, 20036, US

(21) Appl. No.: 10/399,700

(22) PCT Filed: Sep. 9, 2002
(86) PCT No.: PCT/FR02/03054

(30) Foreign Application Priority Data
Sep. 11, 2001 (FR) 01/11719

(51) Int. Cl. A61M 16/00

(52) U.S. Cl. 128/207.14

(57) ABSTRACT

The invention concerns a device comprising a curved tubular body (2), designed to be inserted in the mouth (10) and whereof the curvature corresponds at least approximately to that of the tongue (11). The proximal end of the tubular body (2) is provided with a peripherally projecting stop ring, capable of being positioned on the outside of said patient's incisors (12, 13)

The present invention relates to a device for suppressing snoring by a patient who is susceptible to episodes of sleep apnea.

It is known that snoring is the result of an increase in the flowrate of the air in the upper airways of said patient, this increased flowrate itself being the result of an increase in the resistance to the passage of the air in said upper airways due to the narrowness of these during sleep. This narrowness is a result of insufficient activation of the dilator muscles of the upper airways during sleep.

Thus, in order to cure a patient'snoring, attempts have hitherto been made to restore the tone of said dilator muscles by surgery. This involved performing a major and invasive operation, which did not always have a successful outcome.

The present invention has the object of remedying these disadvantages and it relates to a simple, inexpensive and easy-to-use device which allows the patient using it at night to sleep without snoring.

To this end, according to the invention, the device for suppressing snoring by a patient who is susceptible to episodes of sleep apnea is distinguished by the fact that:

it comprises a curved tubular body which is designed to be introduced into the mouth of said patient, so as to rest at least partially on the tongue of the patient, and of which the curvature corresponds at least approximately to that of said tongue;

the proximal end of said tubular body is provided with a peripherally projecting stop flange which is able to be positioned on the outside of the incisors of said patient; and

the length of said tubular body is such that, when it is in place in the mouth of said patient, the distal end of said tubular body is situated in front of the lingual V of the circumvallate papillae of said tongue.

Thus, by virtue of the present invention, and irrespective of the tone of the dilator muscles of the patient'upper airways during sleep, the passage of air in said airways is guaranteed by said curved tubular body, so that the flow of air cannot assume an excessive flowrate. Snoring is thereby suppressed. Moreover, the presence of the tubular body in the patient'mouth does not cause excessive discomfort or nausea because the distal end of said tubular body cannot reach said circumvallate papillae. Finally, said stop flange ensures that said patient cannot swallow the device. This flange may optionally comprise a covering for purposes of comfort.

Said tubular body can be formed by a tubular element with a solid wall, for example of synthetic material. In this case, in order to further increase the entry and exit of air through said tubular body, the solid wall of said element is advantageously provided with a multiplicity of lateral holes.

Alternatively, the tubular body can be formed, at least for the most part, by a rigid grating, for example also made of a synthetic material.

Irrespective of its configuration, said tubular body preferably has an oblong cross section in order to have a wide flat surface resting on the tongue and thereby reduce the pressure it can exert on the latter, and thus reduce the discomfort which its presence may cause.

To ensure that dust, insects or other undesirable particles inhaled through said tubular body during sleep cannot pass into the patient'upper airways, said tubular body can be surrounded by a net sleeve functioning as a filter. It will be noted that in addition to this filtering function, said sleeve retains the humidity of the patient'exhaled air, which can thus humidify the inhaled air.

To optimize these two functions, said sleeve can be replaced by an open-cell foam material filling said curved tubular body, such a foam material allowing gas to pass through but holding back the solid impurities contained therein.

To allow the device according to the invention to be supported in position in the mouth, said tubular body is shaped, in proximity to said flange, with a support zone which is able to be gripped between the incisors of the lower and upper jaws of the patient. In order to return the device toward the tongue in the event of its moving away from it, the walls of said support zone in contact with said incisors are inclined in such a way that said curved body bears against said tongue when said support zone is gripped between said incisors.

Furthermore, to ensure that a patient'lips cannot inadvertently close off the distal orifice of said device, it is advantageous if, on the proximal side of the stop flange, said curved tubular body is continued by an extension piece projecting from said flange.

The figures in the attached drawing will show clearly how the invention can be realized. In these figures, identical reference numbers designate similar elements.

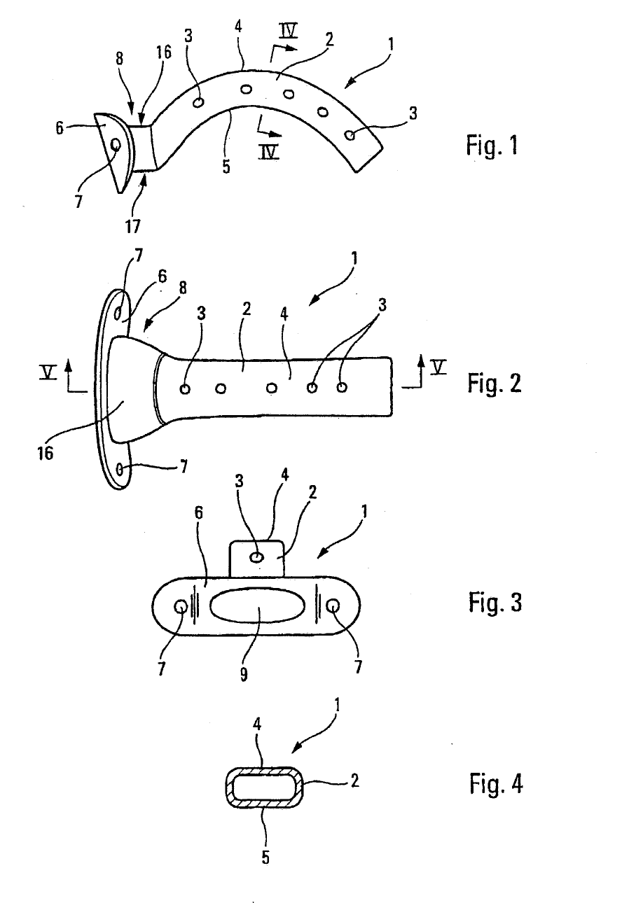

FIG. 1 is a side view of an illustrative embodiment of the device according to the present invention.

FIG. 2 is a plan view and FIG. 3 a front view of the device in FIG. 1.

FIG. 4 is a cross section along the line IV-IV in FIG. 1.

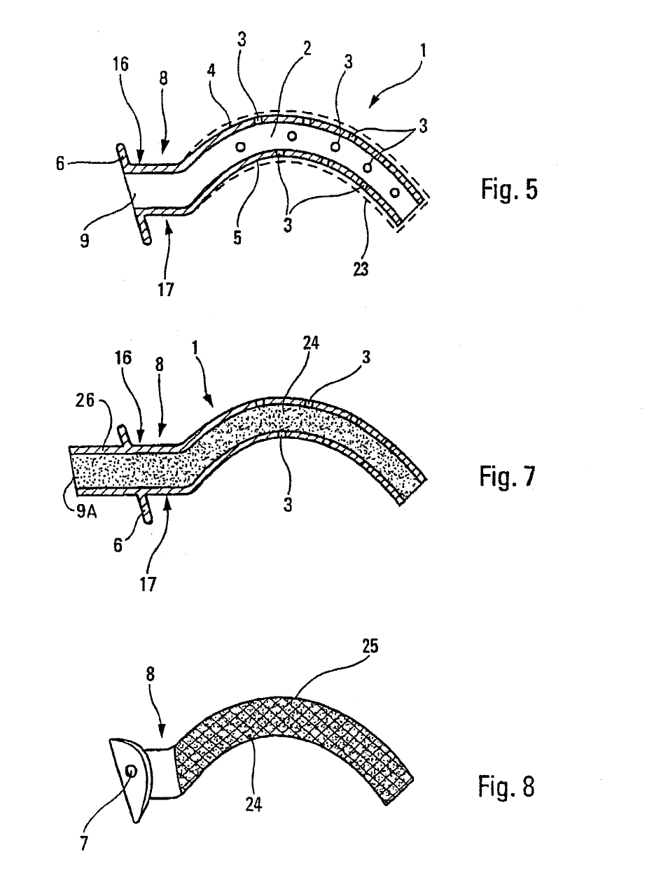

FIG. 5 is a longitudinal section along the line V-V in FIG. 2.

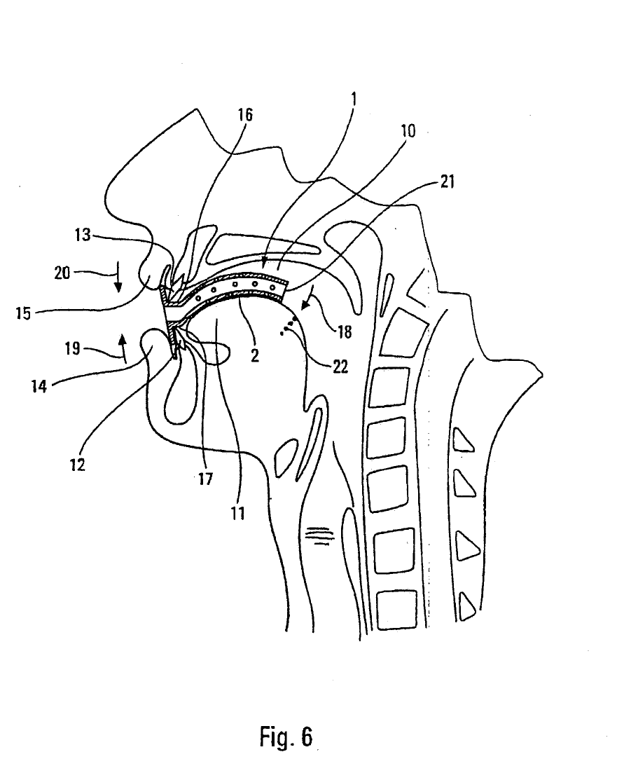

FIG. 6 illustrates diagrammatically the position of the device from FIGS. 1 through 5 in a patient'mouth.

FIG. 7 shows an alternative embodiment of the device of the invention, in longitudinal section similar to that of FIG. 5.

FIG. 8 shows another alternative embodiment of the device of the invention, in a side view similar to FIG. 1.

The device 1 according to the present invention, and illustrated in FIGS. 1 through 5, is intended to suppress snoring by a patient who is susceptible to episodes of sleep apnea, when it is used by said patient during sleep, in the manner illustrated diagrammatically in FIG. 6. This device 1 comprises a curved tubular body 2 with a solid wall provided with a multiplicity of lateral holes 3. The tubular body 2, which is made for example of a synthetic material, has an oblong cross section (FIG. 4), such that its wall has an upper flat surface 4 and a lower flat surface 5.

At its proximal end, the tubular body 2 is provided with a projecting peripheral stop flange 6, itself optionally provided with holes 7 analogous to the holes 3.

Furthermore, in proximity to the stop flange 6, the tubular body 2 is shaped so as to present a support zone 8, for example laterally widened and opening into said flange 6 by way of a wide proximal opening 9.

As is illustrated in FIG. 6, the tubular body 2 is intended to be introduced into the mouth 10 of the patient and to rest at least partially on the tongue 11 of the latter. For this purpose, the curvature of the tubular body 2 corresponds at least approximately to that of the tongue 11. In this position of use, the support zone 8 is gripped by the patient between the incisors 12 of the lower jaw and the incisors 13 of the upper jaw, and said flange 6 is arranged between the lips 14 and 15 of the patient and said incisors 12, 13.

The incisors 12 and 13 bear on the upper face 16 and lower face 17, respectively, of the support zone 8 and these upper 16 and lower 17 faces are inclined in such a way that said curved tubular body 2 bears against the tongue 11 (see arrow 18 in FIG. 6) when said incisors 16, 17 press on said faces 16 and 17 in opposite directions (see arrows 19 and 20 in FIG. 6).

Furthermore, the length of the curved tubular body 2 is chosen so that, in the position of use (FIG. 6), the distal end 21 of said body is situated in front of the lingual V of the circumvallate papillae of the tongue 11.

It will readily be appreciated that, with the device 1 in place, in the manner described above with reference to FIG. 6, in the mouth 10 of a patient who is asleep, there is normal circulation of air between the patient'airways and the outside by way of said device 1, irrespective of the degree of relaxation of the dilator muscles of the upper airways of said patient. The latter'snoring is thereby suppressed.

To ensure that dust, insects, etc. do not penetrate into the upper airways, the tubular body 2 can be surrounded by a net sleeve 23 (see FIG. 5) acting as filter. Moreover, this net 23 retains the humidity of the air exhaled by the patient, which makes it possible to additionally humidify the air inhaled by said patient.

This dual function of filtration and humidification can be still better ensured by a filling 24 of open-cell foam material lodged in said tubular body 2 (FIG. 7).

Alternatively, the tubular body of the device according to the present invention can be made as a rigid grating 25, for example of synthetic material (FIG. 8).

Furthermore, as is shown in FIG. 7, the tubular body 2 can be continued at the proximal end by an extension piece 26 projecting from said stop flange 6 and shifting the proximal opening of said tubular body 2 to an advanced position 9A.

1. A device for suppressing snoring by a patient who is susceptible to episodes of sleep apnea, being a device:
which comprises a curved tubular body (2) which is designed to be introduced into the mouth (10) of said patient, so as to rest at least partially on the tongue (11) of the patient, and of which the curvature corresponds at least approximately to that of said tongue; and
in which the proximal end of said tubular body (2) is provided with a peripherally projecting stop flange (6) which is able to be positioned on the outside of the incisors (12, 13) of said patient,
characterized in that the length of said tubular body is such that, when it is in place in the mouth of said patient, the distal end (21) of said tubular body (2) is situated in front of the lingual V of the circumvallate papillae (22) of said tongue, and in that the lateral wall of said tubular body is openworked.

2. The device as claimed in claim 1, characterized in that the lateral wall of said curved tubular body is solid and is provided with lateral holes (3).

3. The device as claimed in claim 1, characterized in that said curved tubular body consists, at least for the most part, of a rigid grating (25).

4. The device as claimed in one of claims 1 through 3, characterized in that said stop flange (6) is openworked (at 7).

5. The device as claimed in one of claims 1 through 4, characterized in that said tubular body (2) is surrounded by a net sleeve (23).

6. The device as claimed in one of claims 1 through 4, characterized in that said tubular body (2) is filled with an open-cell foam material (24).

7. The device as claimed in one of claims 1 through 6, in which, in proximity to said flange (6), said tubular body (2) is shaped so as to present a support zone (8) which can be gripped between the incisors (12, 13) of the lower and upper jaws of said patient,
characterized in that the walls (16, 17) of said support zone (8) in contact with said incisors (12, 13), respectively, are inclined in such a way that said curved tubular body (2) bears against said tongue (11) when said support zone (8) is gripped between said incisors (12, 13).

8. The device as claimed in one of claims 1 through 7, characterized in that, on the proximal side of said stop flange (6), said curved tubular body (2) is continued by an extension piece (26) projecting from said flange.
